(19) United States
(12) Patent Application Publication
(10) Pub. No.: US 2024/0122613 A1
(43) Pub. Date: Apr. 18, 2024

(54) Thrombectomy Device

(71) Applicant: phenox GmbH, Bochum (DE)

(72) Inventors: Ralf HANNES, Dortmund (DE); Volker TRÖSKEN, Witten (DE); Hermann MONSTADT, Bochum (DE); Hans HENKES, Stuttgart (DE)

(73) Assignee: phenox GmbH, Bochum (DE)

(21) Appl. No.: 18/272,434

(22) PCT Filed: Jan. 11, 2022
(86) PCT No.: PCT/EP22/50381

§ 371 Date: Jan. 4, 2024

(30) Foreign Application Priority Data
Jan. 14, 2021 (DE) 10 2021 100 703.8

(51) Int. Cl. A61B 17/221; A61B 90/00

(52) U.S. Cl. CPC A61B 17/221; A61B 90/39; A61B 2090/3966

(57) ABSTRACT

A substantially tubular structure with a plurality of mesh cells (3, 4) composed of struts, with two connecting struts (5, 5′), disposed at proximal mesh cells at the proximal end of the tubular structure (1) and extending in proximal direction, and being provided with a coupling member (11) disposed proximally of the tubular structure (1) with the connecting struts (5, 5′) being connected to said member, wherein the coupling member (11) being connected to a pusher/insertion wire, and wherein struts of the proximal mesh cells, the connecting struts (5, 5′), and the coupling member (11) as well as bracing struts (9) that may be arranged between the proximal mesh cells form a closed, annular structure (6), and the annular structure (6) comprising a ring marker (12) with a radiopaque material for identifying the spatial position of the annular structure (6).

The invention relates to a thrombectomy device provided with an improved marking of the proximal zone. The thrombectomy device comprises a substantially cylindrical structure, the proximal end of which is connected to a coupling member via connecting struts. The thrombectomy device is specifically designed to remove thrombi located in the cerebral vasculature.

Thromboembolic diseases such as myocardial infarction, pulmonary embolism, peripheral thrombosis, organ embolisms etc. are typically triggered by a thromboembolus (hereinafter referred to as thrombus or clot for short), i.e., a viscoelastic blood clot consisting of platelets, fibrinogen, clotting factors etc., which has lodged in a blood vessel and occludes it completely or partially. The occlusion of organ arteries also leads to an interruption in the supply of oxygen and nutrients to the dependent tissue. The disturbance of the functional metabolism with loss of function is followed within a short time by the breakdown of the structural metabolism resulting in the destruction of the affected tissue (infarction). Organs most frequently affected in this way are the heart and the brain. However, such abnormalities also affect the extremity arteries and pulmonary arteries. Moreover, venous thrombosis and thromboembolic occlusion often occur also in the leg and pelvic veins. The clinical picture of thrombotic occlusion of an intracranial sinus may lead to severe intracerebral hemorrhage due to disruption of venous drainage of brain tissue.

In view of the severity of the clinical picture associated with thromboembolism and the frequency of these conditions various techniques have been developed which are aimed at dissolving or removing thrombi.

Aside from the treatment with thrombolytic agents designed to dissolve the thrombus on the one hand and open surgical interventions to remove the thrombus on the other, microinvasive endovascular forms of therapy are increasingly being applied.

One of these known forms of treatment involves the application of what is known as a thrombectomy device. In WO 2012/156069 A1 such a thrombectomy device has been described having a cylindrical structure with a plurality of mesh cells, as they are also found in vascular stents. The cylindrical structure, which is similar in principle to a known stent structure, comprises two connecting struts that terminate in a coupling member and are connected via the coupling member to a pusher wire that allows precise placement of the thrombectomy device at the treatment site. The coupling member is located at the proximal end of the device and connects it to the distal end of the pusher wire.

In the known thrombectomy device a slot has been arranged that extends in a coiled or helical fashion over the circumferential surface of the cylindrical, tubular structure. Such a thrombectomy device can in particular be fabricated from a tube by laser cutting, but alternative fabrication methods may be adopted, for example making use of interwoven filaments or wires.

The circumferential surface of the cylindrical structure remains open in the area of the slit, so that the diameter of the cylindrical structure is capable of adapting to some extent to the diameter existing at the treatment site, i.e., to the lumen of the vessel.

To hold the slit cylindrical structure securely in position and also bring a certain amount of tension to bear on the structure a bracing clip or strut is used to span the slit at the proximal end of the cylindrical structure. In this way, a circumferentially closed overall structure is produced, which is closed at the proximal end, with the cylindrical structure further distally being provided with a helical slit. The bracing clip increases the radial force of the cylindrical structure in the proximal area and, moreover, serves to keep the oppositely arranged edges of the slit in position relative to each other.

Where the term “proximal” is used herein in connection with the invention, this term refers to the end of the device facing toward the treating person when the device is inserted into the patient's body while the term “distal”, on the other hand, refers to the end of the device facing away from the treating person.

The proximal region of the known thrombectomy device with slit forms a closed annular structure from the bracing clip to the coupling member via struts of the proximal mesh cells and the connecting struts. Even without a slit, such a closed annular structure can be brought about at the proximal end; in this case, however, no bracing clip spanning the slit is required. The annular structure is inclined or oblique with respect to the longitudinal axis of the cylindrical structure, in other words the (approximate) plane that the annular structure forms is diagonal to the longitudinal axis of the cylindrical structure and extends from proximal to distal.

In the interest of achieving optimum functionality of the thrombectomy device, it is in any case of utmost importance that the annular structure opens sufficiently wide at the treatment site and does not contract, or at least does not contract disproportionately, even during the withdrawal of the device.

Only a sufficiently wide-open proximal end enables the thrombus to be successfully ablated with the device. To achieve this, a device would be desirable that allows the treating person to evaluate the three-dimensional state of the proximal annular structure during the intervention. During both the insertion of the thrombectomy device and the retraction of the thrombectomy device such an evaluation or assessment will be most critical to the success of the treatment.

Assistance in the evaluation or assessment during intervention can be provided by X-ray visible markers or radiopaque markers. Markers of this nature have long been known in the field of medical implants and devices, for example in the context of stents and catheter balloons. Appropriate markers are as a rule applied at specific points and mark, for example, the proximal and distal ends of a stent or catheter or special areas or zones of a stent or catheter such as the fenestration in the case of bifurcation implants.

However, the known markers are not suitable for the necessary assessment of the opening behavior or the opening of the proximal annular structure of the thrombectomy device, since they can only inadequately provide information about the spatial state of the annular structure due to their merely point-like attachment. Moreover, known markers may adversely affect the functionality necessary to ablate the thrombus, namely the device's shear forces.

However, since it is especially the proximal section of the thrombectomy device that is of essential importance in the successful removal of the thrombus, it is of greatest significance to obtain most accurate information about the opening state of the annular structure during each step of the intervention. It is, in fact, to be noted that the successful removal of the thrombus out of the vessel depends essentially on the requirement that the device and, in particular, the proximal annular structure have the best possible contact with the vessel wall, as this is the only way to peel the thrombus off the wall of the vessel.

It is thus the objective of the present invention to provide a thrombectomy device provided with radiopaque marking, wherein the radiopaque marking enables the treating person to assess and evaluate the three-dimensional state, in particular of the proximal annular structure, during the intervention without impairing the functionality of the device.

This objective is achieved in accordance with the invention by a thrombectomy device comprising a substantially cylindrical structure having a proximal end and a distal end and having a plurality of mesh cells composed of struts, with the thrombectomy device furthermore comprising two connecting struts, disposed at proximal mesh cells at the proximal end of the cylindrical structure and extending in proximal direction, and being provided with a coupling member disposed proximally of the cylindrical structure to which the connecting struts are connected, with the coupling member being connected to an insertion or pusher wire, wherein struts of the proximal mesh cells, the connecting struts, and the coupling member as well as bracing struts that may be arranged between the proximal mesh cells form a closed, annular structure, and the annular structure comprises a ring marker comprising a radiopaque material for identifying the spatial position of the annular structure.

The thrombectomy device proposed by the invention comprises a substantially cylindrical structure having a proximal end and a distal end, with the cylindrical structure being formed by a plurality of mesh cells. The cylindrical structure is essentially that of a typical stent, but the thrombectomy device serves a different purpose.

The substantially cylindrical structure of the thrombectomy device exhibits a plurality of openings distributed over the circumferential surface, these openings being referred to as mesh cells in accordance with the invention. In other words, a lattice or mesh structure is created, built up of struts, so that a plurality of openings or mesh cells are formed on the circumferential surface of the basic cylindrical structure. Other areas of the thrombectomy device, in particular a proximal section proximally adjoining the cylindrical structure, may also have a lattice structure with openings built up of struts, wherein the diameter of the proximal section as a rule decreases in the proximal direction, so that the proximal section is thus not cylindrical.

At the proximal end of the cylindrical structure, two connecting struts are arranged on (usually different) mesh cells. The connecting struts terminate in a coupling member to which a pusher wire is attached for placement of the device. In the area of the coupling member a proximal marker may be provided, and a distal marker may be arranged at the distal end of the device. The proximal and distal markers may include known radiopaque materials, as they are mentioned below.

A detachment point can be provided on the coupling member or in the area of the pusher wire so that the cylindrical structure can be disconnected if required. However, since, for the purpose of thrombus removal, a thrombectomy device is primarily intended to be withdrawn out of the vascular system, a detachment point is not strictly required; rather, it is only for detachment in the event that the thrombectomy device cannot be retracted. The detachment point can be, for example, an electrolytically corrodible system that allows the cylindrical structure to be disconnected from an insertion/pusher wire. Other detachment methods are known to those skilled in the art, for example mechanical, thermal or chemical release mechanisms.

Optionally, a slit may be arranged to extend helically over the circumferential surface of the cylindrical structure. In this case, a bracing strut, which can also be referred to as a bracing clip, spans the slit at the proximal end of the cylindrical structure.

At least some struts of the proximal mesh cells, the connecting struts and the coupling member together form a closed annular structure. Accordingly, the annular structure is thus created by the relevant struts of the proximal mesh cells, the connecting struts and the coupling member.

If the thrombectomy device is provided with a slit, the bracing strut(s) spanning the slit at the proximal end of the cylindrical structure also form part of the annular structure, i.e., the bracing strut, some struts of the proximal mesh cells, the connecting struts, and the coupling member together constitute the closed annular structure.

As proposed by the present invention, this annular structure comprises at least one ring marker which is disposed on the annular structure in such a way that the marker can provide an indication of the spatial position, resp. the spatial orientation of the annular structure during the intervention and thus allows the user to assess the opening state of the annular structure. The marker of the annular structure will be referred to hereinafter as “ring marker” to distinguish it from other distal and proximal markers. Because the annular structure typically extends diagonally through the lumen of the blood vessel, the ring marker also allows the treating physician to visualize the orientation of the thrombectomy device around the longitudinal axis.

In a preferred embodiment, the ring marker comprises radiopaque material, for example gold, platinum, palladium, tantalum or appropriate combinations based on precious metals or possible alloys and combinations such as platinum-iridium. Radiopaque materials of this kind can also be used for other markers of the thrombectomy device.

Preferably, the ring marker is wire-shaped. Also preferred, the ring marker is provided as a one-piece element, i.e., it comprises a continuous wire.

The wire itself may comprise a plurality of individual strands that are, for example, braided, interlaced, twisted together, or otherwise connected. In accordance with the invention, the wire may thus also be of stranded design. Within the meaning of the invention, a ring marker made of a continuous wire shall be understood such that the wire or, respectively, the ring marker is not interrupted along its length.

The wire-shaped ring marker is preferably wound around the struts that form the annular structure. However, the ring marker may alternatively be arranged on only one side of the annular structure, for example on the inner or outer side, or it may be wound around the annular structure in only one or only a few turns.

It is thought expedient for the ring marker to run around the entire length of the annular structure, that is, starting, for example, from the coupling member or starting from a proximal marker via the first connecting strut and the struts of the proximal mesh cells to the bracing strut, if arranged, and then back via the struts of the proximal mesh cells and the second connecting strut to the coupling member or to the proximal marker.

The ends of the ring marker are preferably secured at the proximal marker or the coupling member.

In alternative embodiments, the disposition of the ring marker may also be limited to only one or more portions of the annular structure, for example, to the bracing strut and/or the connecting struts and/or the struts of the proximal mesh cells. However, in this case as well, the ring marker should adequately delineate the annular structure to such an extent that its unfolding and orientation can be deduced as a whole from visualization using appropriate imaging techniques.

Accordingly, it is also conceivable that the ring marker is composed of a plurality of individual ring marker elements.

Aside from being provided in the form of a wire, the ring marker and ring marker elements may also comprise other shapes and, for example, may be of spiral, tubular, or sleeve-like configuration.

Further embodiments provide for additional markers being placed in position along the cylindrical structure, for example in the central region. These markers as well can be of continuous design or composed of marker elements and provided, for example, in wire form, spiral form, tubular form or sleeve form.

The cross-section of the wire-shaped ring marker and the further markers is preferably round, but embodiments that feature an oval or angular wire are also conceivable.

Preferably, the diameter of the wire ranges between 0.01 and 0.5 mm, respectively, the edge length of the wire is between 0.01 and 0.5 mm.

The attachment of the ring marker or the ring marker elements and of the markers or the marker elements is brought about by known methods, in particular by welding (for example by a laser), crimping, clamping or gluing/bonding.

In addition to the markers referred to hereinbefore, in a further embodiment of the invention it is conceivable to provide for an additional marking of at least parts of the cylindrical structure, i.e., for example, of the proximal, distal or central region, by coating with radiopaque materials. The suitable coating materials are essentially the same as the materials, alloys and combinations hereinbefore mentioned as appropriate for the other markers. For example, a gold coating could be applied.

A thrombectomy device in accordance with the invention may be implemented in various ways. The idea upon which this invention is based, of placing a ring marker along the proximal annular structure is readily transferable to similar devices.

The bracing strut can be subsequently attached to the cylindrical structure; however, this bracing strut may also form part of the structure itself and, for example, be cut together with it from a tube.

The proximal clip in the form of the bracing strut improves the radial force characteristic of the cylindrical structure in the proximal area. In particular, the provision of this clip not only reduces slimming of the cylindrical structure but also the tensile stresses as they occur when the device is retracted into the catheter. At the same time an additional peeling effect is produced same as achieved with the mesh cells and edges of the cylindrical structure.

However, of special significance is that the unfolding force in the proximal zone is improved which enables the cylindrical structure to be optimally adapted to the vessel lumen. At the same time this arrangement prevents areas of cylindrical structure which are separated by the slit from being displaced relative to each other.

To enable the cylindrical structure to be easily drawn into the catheter, the camber of the bracing strut is shaped such that its maximum points toward the distal end of the cylindrical structure. This means the curved portion of the bracing strut is closed distally while toward the proximal end and together with the connecting struts and struts of the proximal mesh cells it forms an annular structure which terminates in the coupling member.

Alternatively, the bracing strut spans the slit in the cylindrical structure in a wave-like fashion, for example in a manner that the bracing strut follows the configuration of the edges of the mesh structure from one side of the slit to the other. In accordance with a variant of the invention, the inventive cylindrical structure may be occluded at the distal end by means of a mesh structure with a view to collecting thrombotic material therein as if using a capturing basket.

The mesh structure of the cylindrical structure may be braided, i.e., built from individual wires or wire strands, but should preferably be a cut structure for which a tube of suitable diameter is used out of which the mesh structure is cut by means of a laser. The material is usually a metal, however plastic material may be employed as well. The elasticity of the material must be sufficient to enable a contraction appropriate to the diameter of a customary catheter and, moreover, produce the expansion to the desired and prescribed diameter when liberated from the catheter. Moreover, it is thought expedient to process the lattice structure by electropolishing to make it smoother and rounder and thus render it less traumatic. This also reduces the risk of germs or other impurities adhering to the structure. The struts or wires may have a round, oval, square, rectangular or trapezoidal cross section, with the edges of square, rectangular or trapezoidal cross sections being advantageously rounded off. Flat struts/wires in the form of thin strips, especially metal strips may be employed as well.

Apart from iron alloys (stainless steel, spring steel) and cobalt-chromium alloys especially shape-memory alloys are suited for use as strut material, for example binary nickel-titanium alloys (nitinol) and ternary nickel-titanium-chromium alloys (chromium-doped alloys). Especially nitinol is known for application in self-expanding structures in the neurovascular field.

In addition to the proximal bracing strut, suitable thrombectomy devices may be provided with further bracing clips in the central and distal regions. However, when using shape memory materials capable of exhibiting an adequate shape recovery effect bracing clips may be dispensed with altogether.

The thrombectomy device is deployed in such a way that it is brought to the site of use by means of a catheter and released either in the thrombus itself, adjacent to or distal to the thrombus. The device expands within the vessel and adapts to the vessel lumen. Either as soon as the device is stretched open or as it is withdrawn, the thrombus material becomes entangled in the mesh structure and is carried into the catheter when the device is withdrawn. Fragments of the clot still adhering to the wall of the vessel are removed and carried away by the shearing action of the mesh and along the edges of the slit. The clot is drawn into the catheter and extracted from the body when the catheter is removed.

By using another catheter, especially an aspiration catheter, additional dissolved particles of the thrombus can be aspirated to minimize the risk of further embolisms that may be caused by these particles.

During extraction of the clot, a helical configuration of the slit across the circumferential surface offers special advantages in that the edges of the cylindrical structure along the slit migrate tangentially along the circumference of the vessel wall during traction. This improves the shearing effect. Moreover, the helical or coiled configuration of the slit also improves (reduces) the bending stiffness to such an extent that the device can better adapt to tortuous vessel patterns, which facilitates both the placement of the device and the extraction of clots from complex vascular structures.

As mentioned hereinbefore, the inventive cylindrical structure is preferably cut out of a cylindrical tube with the help of a laser. Using this method enables the individual mesh cells to be provided with special cross sections, for example square, rectangular or trapezoidal cross sections. In case of rectangular and trapezoidal shapes, either the small/short side of the cross section may be located peripherally, or the longer side. It is preferred, however, that the narrow side of both the rectangle and, in particular, the trapezoid faces the vessel wall which allows easier penetration of the thrombus into the mesh structure and permits the clot mass to be effectively displaced when the cylindrical structure expands.

The connecting struts located at the proximal end of the cylindrical structure extend from the proximal mesh cells adjoining the slit to a coupling member where they are brought together. They form part of the cylindrical structure and for that reason usually are built from the same material.

Further elucidation of the invention is provided by way of examples through the enclosed figures. It is to be noted that the figures show preferred embodiment variants of the invention, with the invention itself not being limited thereto. To the extent it is technically expedient, the invention comprises, in particular, any optional combinations of the technical features that are stated in the claims or in the description as being relevant to the invention. Clarification of the invention is provided by the following figures where

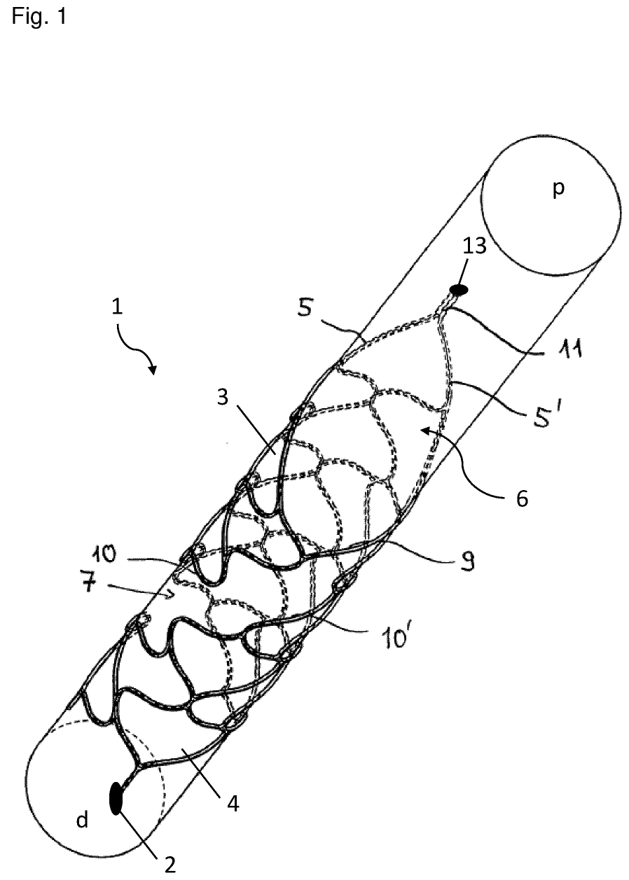

FIG. 1 is a spatial representation of the known cylindrical structure;

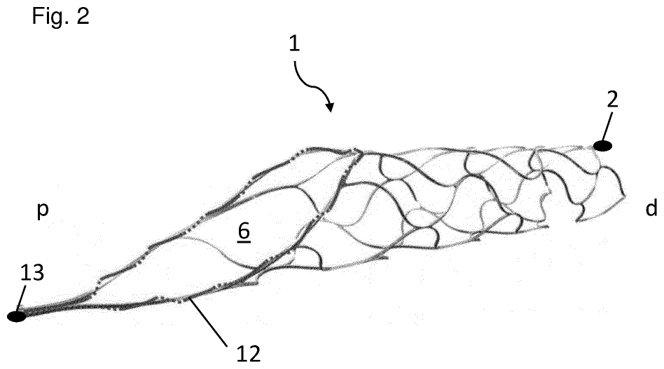

FIG. 2 is a spatial representation of the preferred embodiment of the device according to the invention.

FIG. 1 shows a spatial representation of a variant of the known cylindrical structure 1 with preferably differently designed mesh cells 3, 4 and the connecting struts 5 and 5′. Mesh cells 3, 4 give stability and flexibility to the overall structure.

A slit 7 runs through the cylindrical structure 1, with said slit being bridged by the bracing strut 9 at the proximal end of the cylindrical structure 1. Slit 7 is delimited by edges 10 and 10′ of the mesh structure. The slit 7 preferably runs obliquely to the longitudinal axis of the cylindrical structure 1, which is represented in the spatial view as a helical extension along the circumferential surface.

The struts of the cylindrical structure 1 located on the rear side of the cylindrical structure 1 are shown in lighter color. It can be seen that at the proximal end of the cylindrical structure 1 slit 7 is located that passes under bracing clip 9 and extends in a helical fashion to the right around the circumferential surface of the cylindrical structure 1. The slit 7 ends distally on the underside of the cylindrical structure 1, thus describing a rotation/turn of approximately 180°.

The connecting struts 5, 5′ are brought together in a single coupling member 11, via which there is a connection to the pusher wire, which is only hinted at here.

A proximal marker 13 is provided in the area of the coupling member 11, indicating the proximal end of the cylindrical structure 1.

Slit 7 is bridged by the bracing strut 9. Bracing strut 9 attaches to the mesh cells 3 located at the edges 10, 10′ of the mesh construct and said strut points with its arc to the distal side of the cylindrical structure 1, which allows easy retraction of the cylindrical structure 1 into a catheter.

In a preferred embodiment, the bracing strut 9 can take up the course of the side surface or edge 10 of the mesh structure with its undulating course and continues this to the oppositely located edge 10′. Alternatively, however, the bracing strut 9 can also take a simply arched, that is, non-wavy course.

The connecting struts 5, 5′ with the adjoining mesh edges and the bracing strut 9 jointly form a kind of loop similar to the opening of a capturing basket which facilitates retracting the thrombectomy device into a catheter and moreover is suited to shear off clots or clot remnants adhering to vessel walls.

Together with the coupling member 11, the connecting struts 5, 5′, the mesh edges adjoining them and the bracing strut 9 form an annular structure.

The distal end of the cylindrical structure may additionally be closed off by means of a mesh structure.

FIG. 2 shows the device proposed by the invention, with a ring marker 12 being provided along the proximal annular structure 6. In the preferred embodiment as illustrated, the ring marker 12 comprises a wire-like radiopaque material that is wound in a loop-like fashion around the entire circumference of the annular structure 6. Preferably, the wire-shaped ring marker 12 is provided as a one-piece element, thus comprising only a single continuous wire. The wire itself may comprise a plurality of individual strands that are, for example, braided, interlaced, twisted together, or otherwise connected. According to the invention, a wire-shaped ring marker 12 made of a continuous wire is thus a ring marker 12 which is not interrupted along its length, that is, is provided as a single one-piece element.

The ends of the ring marker 12 are secured in or on proximal marker 13.

Additional proximal markers 13 and distal markers 2 may be provided.

In the figures identical reference numerals are meant to refer to the same subject matter.

### LIST OF REFERENCE NUMERALS

1 Cylindrical structure
2 Distal marker
3, 4 Mesh cells
5, 5′ Connecting struts
6 Annular structure
7 Slit
9 Bracing strut
10 Mesh edges
11 Coupling member
12 Ring marker
13 Proximal marker
d Distal
p Proximal

1. A thrombectomy device comprising a tubular structure (1) having a proximal end and a distal end and comprising a plurality of mesh cells (3, 4) composed of struts, including
two connecting struts (5, 5′) arranged at proximal mesh cells at the proximal end of the tubular structure (1) and extending in the proximal direction, and
a coupling member (11) arranged proximally of the tubular structure (1), with connecting struts (5, 5′) being attached to said member, and said coupling member (11) being connected to an insertion or pusher wire,
wherein struts of the proximal mesh cells, the connecting struts (5, 5′) and the coupling member (11) as well as a bracing strut (9) possibly arranged between the proximal mesh cells form a closed, annular structure (6), wherein
the annular structure (6) comprises a ring marker (12) with a radiopaque material for visualizing the spatial position of the annular structure (6).

2. A thrombectomy device according to claim 1, wherein a slit (7) extends in a helical or coiled fashion over the circumferential surface of the tubular structure (1), with a bracing strut (9) spanning the slit (7) at the proximal end of the tubular structure (1).

3. A thrombectomy device according to claim 1, wherein the ring marker (12) encompasses the annular structure (6) at least partially along its length.

4. A thrombectomy device according to claim 3, wherein the ring marker (12) encompasses the annular structure (6) along its entire length.

5. A thrombectomy device according to claim 1, wherein the ring marker (12) is wire-shaped or sleeve-shaped and wound or laid around the struts forming the annular structure (6).

6. A thrombectomy device according to claim 5, wherein the wire-shaped ring marker (12) is of one-piece design.

7. A thrombectomy device according to claim 1, with a proximal marker (13) in the region of the coupling member (11).

8. A thrombectomy device according to claim 1, wherein the ends of the wire-shaped ring marker (12) are secured in or to the proximal marker (13) or coupling member (11).

9. A thrombectomy device according to claim 1, wherein the bracing strut (9) forms a camber, with the maximum of the camber pointing toward the distal end of the tubular structure (1).

10. A thrombectomy device according to claim 1, with further distal or and/or proximal markers made of a radiopaque material.

11. A thrombectomy device according to claim 1, wherein the radiopaque material is selected from gold, tantalum, platinum, palladium or an alloy based on precious metals or a combination of these materials such as platinum-iridium.

12. A thrombectomy device according to claim 1, wherein further markers are provided along the tubular structure (1), and these markers may be provided in wire form, spiral form, tubular form, or sleeve form.

13. A thrombectomy device according to claim 1, wherein additional marking of at least parts of the tubular structure (1) is provided by coating with radiopaque materials.
